(19) United States
(12) Patent Application Publication
(10) Pub. No.: US 2010/0075921 A1
(43) Pub. Date: Mar. 25, 2010

(54) Prêparation of hyaluronic acid with desired low molecular weight as a food supplement

(76) Inventor: Ahmad Alkayali, Murrieta, CA (US)

Correspondence Address: Law Office of Terry L. Miller, 24832 Via San Fernando, Mission Viejo, CA, 92692, US

(21) Appl. No.: 12/592,504

(22) Filed: Nov. 25, 2009

Related U.S. Application Data
(63) Continuation-in-part of application No. 11/057,882, filed on Feb. 15, 2005.

(51) Int. Cl. A61K 31/728; C08B 37/08; C12P 19/26

(52) U.S. Cl. 514/54; 536/53; 435/84

(57) ABSTRACT

A process for preparing hyaluronic acid primarily as a food supplement which is capable of absorption and assimilation by the human body includes desirable control of both the purity and molecular weight range of the resulting product. Chicken comb tissue is subjected to one of two disclosed processes for extracting, purifying, and controlling the molecular weight range of hyaluronic acid in solution, which is then dried and powdered to a form suitable for human consumption as a food supplement. The resulting hyaluronic acid product can also be used topically in creams or solutions for beneficial treatment of skin conditions, such as dry skin or wrinkling, for example.

### CROSS REFERENCE TO RELATED APPLICATION

This is a Continuation-In-Part of U.S. application Ser. No. 11/057,882, filed 15 Feb. 2005.

### FIELD OF THE INVENTION

The present invention is in the field of preparing a food supplement consisting of hyaluronic acid (or a salt thereof—hereinafter “HA”) in a substantially pure form and within a desired and sufficiently low molecular weight range so that the product is especially suitable and beneficial for oral consumption by humans as a food supplement. That is, the HA product must have a sufficiently low molecular weight that it is absorbed and can be put to beneficial use, when ingested orally by a human. Surprisingly, the Applicant has discovered a process whereby HA may be extracted from chicken comb, purified, and reduced to the desired low molecular weight range without the use of bleach (i.e., Sodium hypochlorite) and in a single process. That is, the prior art would suggest that HA must first be extracted from a source in a first process, and that the product of this first process must be further processed (i.e., in a second separate process) to reduce the molecular weight of the HA for various uses. Even then, the prior art does not teach an extraction, purification, and MW reduction process which will provide a food product HA as is provided by the present invention. The art teaches a number of extraction processes, but not one using only water soaking to obtain a solution of extraction from a source material and including HA.

The inventive produce has both a desirable molecular weight range allowing it to be absorbed, assimilated, and utilized by the human body, and a purity and presentation of useful hyaluronic acid making the inventive product useful as a food supplement for humans. The hyaluronic acid product can also be utilized in creams or solutions for beneficial topical application, as an assistance in alleviating dry skin conditions, for example.

### BACKGROUND OF THE INVENTION

Hyaluronic acid is a mucoid polysaccharide of biological origin, which is widely distributed in nature. For example, it is known that hyaluronic acid is present in various animal tissues such as umbilical cord, synovial fluid, vitreous humor, chicken rooster and chicken hen comb, egg shell membrane, and various connective tissues such as skin and cartilage.

Chemically, hyaluronic acid is a member of glycosaminoglycans and it is constituted by alternating and repeating units of D-glucuronic acid and N-acetyl-D-glucosamine, to form a linear chain having a molecular weight up to 1.3×10⁷ Daltons. Pharmaceutical, ophthalmic, and surgical uses of hyaluronic acid or of a salt thereof is widely described in the literature. For example, hyaluronic acid as a non-immunogenic substance has viscoelastic and hydrophilic properties, and it is used as an eye vitreous or joint fluid replacement or as a supportive medium in ophthalmic surgery, as disclosed for example in U.S. Pat. No. 4,141,973 of Balazs. In joint fluids, the viscous hyaluronic acid solution serves as a lubricant to provide a protective environment to the cells, and for this reason, it is used in the treatment of inflamed knee joints.

Hyaluronic acid of high molecular weight for pharmaceutical, surgical, and ophthalmic uses, for example, can be extracted and purified from various sources, including from umbilical cords, from chicken rooster combs, from chicken hen comb, or from group A and C Streptococci as disclosed for example in U.S. Pat. No. 4,141,973 of Balazs and U.S. Pat. No. 5,559,104 of Romeo et al. Production of hyaluronic acid by Streptococci was first disclosed by Forrest et al. in 1937, (J. Biol. Chem. 118, 61 (1937)) and later it was demonstrated that hyaluronic acid from animal source is identical to hyaluronic acid from microbial source. The biosynthesis of hyaluronic acid by Streptococci is disclosed for example in U.S. Pat. No. 4,897,349 of Swann et al. A number of processes are known for obtaining pharmaceutical grade hyaluronic acid or a sodium salt thereof from microbial sources. For example, U.S. Pat. No. 4,780,414 of Nimrod et al., U.S. Pat. No. 4,517,295 of Bracke et al., and U.S. Pat. No. 5,563,051 of Ellwood et al. disclose processes for obtaining hyaluronic acid by continuous fermentation of Streptococcus bacteria and then purifying hyaluronic acid thus obtained up to pharmaceutical grade.

It is believed, however, that the ability of the human body to synthesize hyaluronic acid as part of the natural body tissues decreases with advancing age. Accordingly, it is also believed that the supplementation of the diet with a form of hyaluronic acid that can be absorbed, assimilated, and utilized by the human body is increasingly more beneficial as age advances. Further, the ability of the human body to absorb and assimilate hyaluronic acid via the digestive tract is believed to rule out the use as a food supplement of many (if not all) of the known hyaluronic acid products currently available. That is, the high molecular weight of these products will result in their simply passing through the digestive system without absorption, assimilation, or digestion.

Several HA extraction or reduction (i.e., decomposition of molecular weight) processes are known in the art. For example, Callegaro (U.S. Pat. No. 6,232,303) appears to relate to a process for making lower molecular weight Hyaluronic acid (HA) from higher molecular weight HA. Callegaro does not teach how the higher molecular weight HA is to be obtained. Callegaro states (column 1, line 54 through column 2 line 6) that there are many examples of preparation of HA fractions by methods involving heating, ultrasounds, UV and gamma irradiation, or by enzymatic reactions (i.e., hyaluronidase), or by chemical depolymerizing reactions with ascorbic acid, or by treatment with hypochlorites. Callegaro states that all of these methods are flawed in some way, and states that they are unable to generate degraded, low-molecular weight product. In fact, Callegaro goes on to state that depolymerization induced by the action of hyaluronidase does not guarantee products characterized by low molecular weight distribution (column 2, lines 7-14).

Callegaro teaches to use bleach at a concentration believed to be at least 14% (by volume, this is over 7000 PPM) in combination with ultrasound to obtain a lower molecular weight HA product. That bleach concentration used by Callegaro is a much, much higher concentration than is authorized by the FDA for use in processing food. Accordingly, the product made by Callegaro could not be used for human oral consumption. Particularly, Callegaro teaches that bleach is to be used in simultaneous combination with ultrasound to process the HA material. According to Callegaro's own examples, use of bleach alone, or use of ultrasound alone (or in sequence) is found to not be a viable process (See, Callegaro at column 4, where the methods designated A, B, and C, are described). At column 5, lines 49-67, Callegaro points out that the methods A and B do not work satisfactorily. Callegaro teaches that method C, using the simultaneous action of bleach and ultrasound is the workable embodiment of the invention. Despite this fact Callegaro includes a puzzling statement at column 8, lines 41-43,asserting that the HA product “could be used in industry, foodstuffs, and cosmetics,” Callegaro is not specific as to what this assertion concerning use of the HA product in foodstuffs means, however, this use would be contrary to FDA guidelines.

Della Valle, EP publication 0138572, appears to relate exclusively to production of HA as a pharmaceutical product having a high molecular weight, and not at all related to production of an HA food supplement, which must have a lower molecular weight in order to be absorbed and utilized by the human body when ingested.

Romeo, WO 92/18543 is for the production of a high molecular weight HA for medical uses, and is not for the production of a low molecular weight HA product for human oral consumption. Generally, the process includes extraction using an organic solvent, then protein is removed using enzyme in the presence of a buffer, the solution is filtered and sterilized, and the purified fraction is precipitated using an organic solvent such as alcohol (Romeo pages 8 and 9). There is no reduction of the HA to a low enough molecular weight range that it could be used as a food supplement.

Balazs, U.S. Pat. No. 4,141,973 is addressed to producing a high molecular weight HA product for medical applications (i.e., for ocular uses). Balazs does not teach a method of processing Hyaluronic acid including decomposing the HA to provide a food supplement. Balazs does teach the use of an enzyme in the process, but this enzyme use is for extraction of the HA, not for the purpose of reduction of the molecular weight of the HA product to a molecular weight range suitable for a food supplement. In fact, Balazs wants a very high molecular weight HA product, as is pointed out in the abstract of Balazs. Balazs states the HA product is to have a molecular weight range from 750 kD to 1,200 kD. This molecular weight is much, much too high for use of the HA product as a food supplement. In contrast, the desired and claimed molecular weight range of the present invention is much lower (i.e., 8 kD to 48 kD). There is no teaching or suggestion in Balazs that a lower molecular weight HA product can be obtained, or that a food product may be produced by the process of Balazs. Balazs reference teaches that HA has a molecular weight from about 50 kD to 8000 kD, although reports exist of HA with a molecular weight of 13,000 kD. Balazs addresses only high molecular weight HA product for medical applications (i.e., ocular uses), and does not at all address HA of a sufficiently low molecular weight that it would have any use as a food supplement. Balazs, is addressed exclusively to producing a very high molecular weight (greater than 50,000 Daltons). Balazs does state that the HA product can be used “in the body,” but this is not a food use, it is a medical injected or inserted use (i.e., as a substitute for or supplement to the aqueous humor of the eye, for example). “Use in the body,” by Balazs does not mean “oral consumption.”

Scott, EP 0295092 is addressed to a topical HA product for application to the skin, which is asserted to promote healing. This HA product of Scott is not a food supplement. Scott teaches a product for topical application (i.e., on the body) including HA fragments of unspecified molecular weight. While this reference states that the fragments of HA include polysaccharides containing from 7 to 50 monosaccharides, this reference does not state that the molecular weight range is from about 1,500 to about 10,500, or any other molecular weight or weight range. Scott simply states that the fragments of HA include polysaccharides containing from 7 to 50 monosaccharides, and that the HA fragments additionally have preferred termination unit chemistry. Scott does not state a molecular weight or weight range for the HA fragments, it is believed, and they could have a much higher molecular weight than the range asserted above. Again, Scott does not state a molecular weight range for the HA product, and this weight range is left uncertain by the teaching of Scott. Specifically, Scott at page 2, lines 55-57 states the above-noted molecular structure, but does not state a molecular weight range.

Udell, US patent publication 2003/0119781 appears to teach that HA can be orally administered in a gelatin formulation. However, Udell specifically says that his “low molecular weight” HA in fact has a molecular weight of 50 kD to 200 kD, which is much too HIGH a molecular weight for effective use as a food supplement. Especially in comparison to the claimed molecular weight ranges of the present invention, the product of Udell is much too high in molecular weight. Udell teaches a so-called “low molecular weight” HA product for oral administration that is in fact higher in molecular weight than the top or highest end of the Applicant's teaching for a food supplement HA product. It is thus believed that the HA product of Udell would not be of any real use as a food supplement, as the human body could not absorb this HA product of Udell when ingested.

The invention of the present application asserts a range of 8000 to 15,000 Daltons as recited by the present claims. Further, the described extraction and purification procedures of the conventional art are complex and result in high production costs, and in a product of a molecular weight range too high for use of the product as a food supplement for humans. The art does not teach a simple water-soaking extraction, and a process of molecular weight reduction process which is free of bleach, yielding a product that is also completely free of bleach.

### SUMMARY OF THE INVENTION

An object of the present invention is to obtain a hyaluronic acid (or a salt thereof) from chicken comb (either from rooster comb or from hen comb) which has sufficient purity for use as a food supplement for humans, and which also has a desired low molecular weight range, making the hyaluronic acid product available for absorption and assimilation via the digestive tract of humans for beneficial use in the human body as a food supplement.

According to one particularly preferred embodiment of the present invention, a method for providing a salt of hyaluronic acid (HA) with a desired molecular weight range includes steps of: providing a solution of extraction including HA and undesired proteins; removing a substantial fraction of said undesired proteins from said solution of extraction; decomposing the HA to a desired low molecular weight range; separating said decomposed HA; and drying the decomposed HA.

Other objects, characteristics, and advantages of the present invention will be apparent from a reading of the following detailed description to two particularly preferred exemplary embodiments of the invention.

### BRIEF DESCRIPTION OF THE DRAWING FIGURES

FIG. 1 provides a process flow chart illustrating the steps in a first preferred exemplary method of preparing hyaluronic acid as a food supplement from rooster comb; and

FIG. 2 provides a process flow chart illustrating the steps in a second preferred exemplary method of preparing hyaluronic acid as a food supplement from hen comb.

### DETAILED DESCRIPTION OF PREFERRED EXEMPLARY EMBODIMENTS OF THE INVENTION

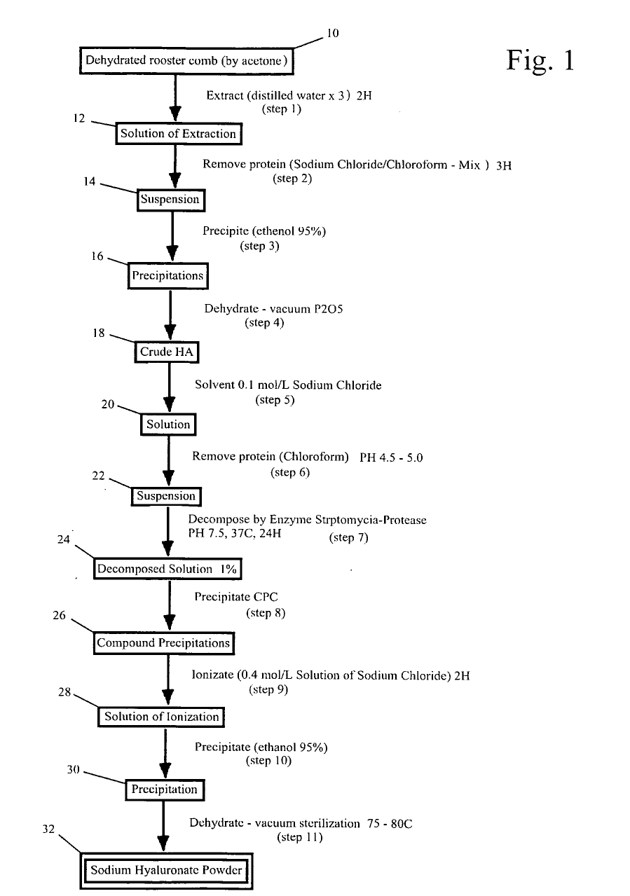

Considering first FIG. 1, it is seen that a process of preparing Hyaluronic Acid (hereinafter, “HA”) as a food supplement suitable for beneficial consumption by humans, according to this particularly preferred embodiment, begins (as is indicated with the numeral 10) with ground dehydrated rooster comb. That is, the raw rooster comb is harvested as part of the chicken slaughtering process, and is dehydrated using acetone and then is ground to a particle size of about 1 to 2 mm.

This dried (i.e., dehydrated) rooster comb 10 is then subjected to an extraction step (Step 1, preferably repeated three times) in which the dehydrated rooster comb is soaked in distilled water for an interval of about 2 hours. Surprisingly, this water-soaking step provides effective extraction of HA from the source material without the use of enzymes, and without the use of other extraction expedients known in the art. As mentioned, it is preferred that this soaking extraction step be repeated three times, although the invention is not so limited. The resulting batches of aqueous solution are combined, to produce a Solution of Extraction 12. This Solution of Extraction contains both the desired HA and undesired proteins which have also been placed into solution during the soaking step.

Undesirable proteins which have been put into solution by the soaking in distilled water are partially removed from the Solution of Extraction 12 using sodium chloride and chloroform and mixing for an interval of about 3 hours (Step 2). This step 2 results in a Suspension 14, to which ethanol is added to cause precipitation (Step 3). The resulting Precipitations 16 (separated from the solution) are dehydrated using vacuum (Step 4) to produce a Crude HA product 18.

Next, the Crude HA product 18 is dissolved (Step 5) using a sodium chloride solvent (i.e., aqueous solution), preferably at a 0.1 mol/L concentration. This step 5 produces a Solution 20, from which proteins are removed (Step 6) by use of chloroform at a PH of about 4.5 to 5.0. This step 6 produces a suspension 22, in which the HA is decomposed to a desirably low molecular weight range (Step 7), using enzymatic digestion. That is, Strptomycia-protease enzyme is used at a PH of about 7.5, for an interval of about 24 hours, at a temperature of about 37° C., to produce a Decomposed Solution 24. As will be further explained below, the temperature and time interval of this digestion step 7 is varied in order to produce an HA product having a desired molecular weight range so that the HA product is available for absorption via the digestive tract of a human. Surprisingly, this enzymatic decomposition step has been found to provide a reliably low molecular weight HA product. In view of the fact that human production of HA for use in various body tissues is believed to decrease with age, it is believed also that health, flexibility of joints and elasticity of various tissues, as well as other beneficial results will be realized in the human body by the ingestion of HA in a molecular weight range as set forth herein.

The Decomposed Solution 24 will contain about 1% of HA, as is noted on FIG. 1. Next, the Decomposed Solution 24 is precipitated (Step 8) and the solvent is removed to produce a Compound Precipitations 26. Next, the Compound Precipitations 26 is dissolved (Step 9) and is also ionized by use of a solvent of 0.4 mol/L sodium chloride in water, soaking the Compound Precipitations for an interval of about 2 hours.

This preceding step 9 provides a Solution of Ionization 28 which is precipitated using 95% ethanol (Step 10), and the solvent is separated, to provide a resulting Precipitation 30, which is saved. The Precipitation 30 is then dehydrated using vacuum sterilization (Step 11) at a temperature of about 75 to 80° C. to produce the final Sodium Hylauronate powder 32.

Having considered the steps of a method of providing Sodium Hyaluronate powder 32, as outlined above, attention may now be given to the details of Step 7, by which a desired selective control of the molecular weight range of the product HA is exercised. That is, by selective control and variation of the time and/or temperature of the enzymatic digestion step 7, the molecular weight range of the resulting HA powder product 32 is controlled. Desirably, the HA powder 32 has a molecular weight range of from about 8,000 to about 15,000 Daltons. More desirably, the HA powder 32 has a molecular weight range of from about 8,000 to about 12,000 Daltons or lower. As described above, this molecular weight range for the HA product 32 is controlled by selective control of the enzymatic digestion or decomposition step 7. That is, variations of the combination of time and temperature used for this step 7 will result in variations of the molecular weight range for the HA product 32 so that the desired molecular weight range is achieved.

Once the HA powder product is obtained, it may be packaged for distribution and use as a human food supplement. This packaging may include, for example, simply providing the powdered HA product in a bottle or jar for consumption with food or drink, or the packaging of the powder HA product into gelatin capsules for ease of swallowing, as is further explained below.

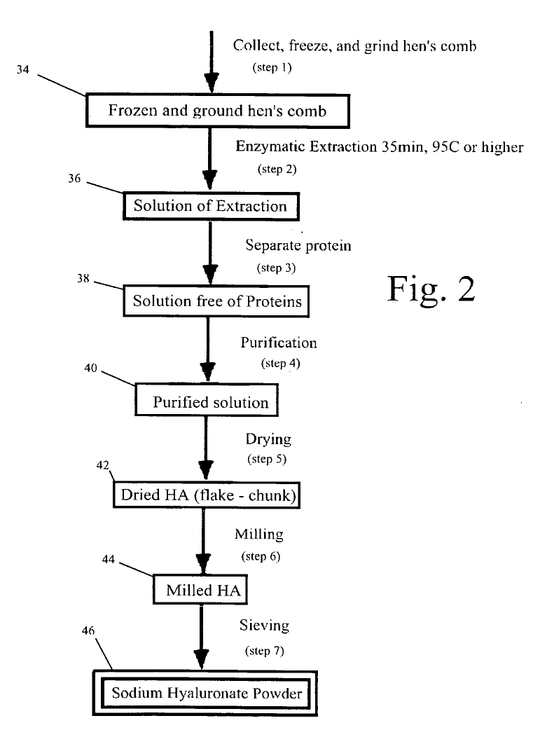

Turning now to FIG. 2, an alternative method of providing a HA food supplement product is illustrated. Viewing FIG. 2, it is seen that in this embodiment, the preferred starting material is chicken hen's comb. The hen's comb is collected as part of the slaughtering process, and is then frozen, and ground (step 1) to provide a collected, frozen and ground starting material 34. Most preferably, the material 34 has a granule size of from about 1 mm to about 2 mm. At step 2 of this process, the starting material 34 is subjected to an enzymatic extraction step (step 2) using water as the solvent and for a time interval and at a temperature that are respectively shorter and higher than was the case with step 1 of FIG. 1. However, this step 2 of the method illustrated in FIG. 2 also includes an enzymatic digestion action (i.e., decomposition action), for which the same enzyme used in the method of FIG. 1 may be used. That is, the Strptomycia-protease enzyme may be used in the water used for this extraction step. The result is a Solution of Extraction 36, which includes both the desired HA and undesired proteins also extracted from the starting material 34. However, because the Solution of Extraction 36 has been subjected to an enzymatic digestion action simultaneously with the extraction from the comb tissues, the molecular weight range of the resulting HA product will be controlled by selective variation of the time, temperature, and enzyme activity (i.e., by choice of enzyme, and enzyme concentration). In this sense then, the Solution of Extraction 36 is also a solution of decomposition or reduction (i.e., reduction in molecular weight of the HA).

Next, the Solution of Extraction 36 is subjected to a step (step 3) to separate undesired proteins from the Solution of Extraction. As described above with reference to the method of FIG. 1 (i.e., at step 6 of that method), the undesired proteins may be removed by the use of chloroform. The PH of the Solution of Extraction is controlled during the exposure to chloroform to effect the desired separation of the undesired proteins from the Solution of Extraction. The result is a Solution Free of Proteins 38. This Solution Free of Proteins is further purified (step 4) to provide a Purified Solution 40.

Then, by use of vacuum drying, a Dried HA product 42 is obtained. This product 42 will be in the form of flakes or chunks, dependent upon such factors as the particulars of the drying process, the equipment used, and the concentration of the HA in the Purified Solution 40. Next, the product 42 is subjected to milling (step 6—milled HA is intermediate product 44) and to sieving (step 7) to provide a milled and sieved sodium hyaluronate powder 46.

Again, by selective control and variation of the time and/or temperature of the enzymatic extraction step 36, the molecular weight range of the resulting HA powder product 32 is controlled. For this process also, the HA powder 48 has a molecular weight range of from about 8,000 to about 15000 Daltons. More desirably, the HA powder 48 has a molecular weight range of from about 8,000 to about 12,000 Daltons or lower.

As described above, this molecular weight range for the HA product 48 is controlled by selective control of the enzymatic extraction step 36 That is, variations of the combination of time and temperature used for this step 36 will result in variations of the molecular weight range for the HA product 48 so that the desired molecular weight range is achieved. And again, once the HA powder product is obtained, it may be packaged for distribution and use as a human food supplement. This packaging may include, for example, simply providing the powdered HA product in a bottle or jar for consumption with food or drink, or the packaging of the powder HA product into gelatin capsules for ease of swallowing. As an example of such packaging of the HA powder into gelatin capsules, a preferred capsule size is from 50 to 100 mg capsules, with an adult daily dosage of 100 to 200 mg. Alternatively, the capsule size may be from 100 mg to about 150 mg, with the number of capsules per day adjusted accordingly. Or, still alternatively, the capsule size may be from about 200 mg to about 250 mg, and the daily dosage would be one or two capsules once or twice per day for an adult human as a food supplement.

In view of the above, it will be appreciated that the present invention may be subject to many alterations and variations without departing from the spirit and scope of the appended claims, which alone define the invention. That is, the starting material for the method of FIG. 1 is chicken rooster comb, while the starting material for the method of FIG. 2 is chicken hen's comb. However, the method of FIG. 1 could be used to extract HA from hen's comb, while the method of FIG. 2 could be used to extract HA from rooster comb. In each instance a desirable HA food supplement product could be obtained, even though the particulars of the method of both FIGS. 1 and 2 are optimized for rooster comb and for hen's comb, respectively. A surprising result follows in the first instance in that merely water-soaking the source material (i.e., chicken comb) according to the process of FIG. 1 is effective to provide a solution of extraction containing HA.

Further, another surprising result follows in that enzymatic decomposition (or reduction of molecular weight) according to the processes of FIGS. 1 and 2 can be used to reliably obtain an HA product of desired low molecular weight. Clearly, the prior art as indicated by the Callegaro reference in particular, would indicate that this is not possible. And, finally, another surprising result is obtained by use of this present invention, in that HA of desired low molecular weight can be obtained without the use of bleach in the process. As a result, the HA product produced by use of this invention is free of bleach. As pointed out above, the low molecular weight HA obtained by use of this invention is intended primarily for use as a food supplement. As such, the use of bleach in the process is at least undesirable, and may in fact be barred by FDA regulations. Further, it will be recognized that the teachings of this invention could be used to obtain HA as a food supplement product from staring materials other than chicken comb (i.e., from materials other than rooster comb or hen's comb).

Moreover, the teachings of this invention could be used to extract a food supplement HA product from, for example, turkey comb. While the tissues of turkey comb may differ somewhat from rooster comb (e.g., possibly having a higher constituent of sodium than is the case with rooster comb), this difference in the starting material can be compensated for by using a slightly less salty concentration of sodium chloride at step 2 of the method of FIG. 1, for example. By thus adjusting the particulars of the methods of FIGS. 1 and 2 as taught by this invention, HA as a food supplement material can be obtained from a variety of starting materials. Importantly, it will be noted that the solutions initially obtained from the starting materials according to this invention contain both extracted HA, and undesired proteins also obtained from the starting materials (i.e., from the rooster comb tissue, for example). According to the teaching of this invention, although the undesired proteins are also extracted along with the desired HA, these proteins are subsequently removed and are separated from the HA, so that a final HA food product of sufficient purity for human consumption is obtained. Also, very importantly, the HA food supplement product obtained according to the teaching of this present invention has a desirably low molecular weight range to allow its oral consumption by a human, with beneficial assimilation and utilization by the human body.

I claim:

1. A method of producing Hyaluronic acid (HA) in the form of a salt of HA which is completely free of bleach and has a desired average molecular weight range from about 8,000 to about 15,000 Daltons, and of utilizing said HA as a food supplement for beneficial human oral consumption as a dry powder, said method comprising steps of:
providing a solution of extraction including HA and undesired proteins;
removing a substantial fraction of said undesired proteins from said solution of extraction;
decomposing the HA in solution to a desired low average molecular weight range from about 8,000 to about 15,000 Daltons without the use of bleach, thus allowing for beneficial human oral consumption as a food supplement;
separating said decomposed HA from solution; and
drying the decomposed HA to a dry powder form for human oral consumption as a food supplement; and
providing the dry powder form of HA to a human as a food supplement free of bleach for human oral consumption.

2. The method of claim 1 including providing said solution of extraction by aqueous extraction from chicken comb, which aqueous extraction includes soaking said chicken comb substantially only in water.

3. The method of claim 2 including the step of utilizing chicken rooster comb.

4. The method of claim 3 further including the steps of:
utilizing dehydrated, granulated rooster comb and soaking said rooster comb in distilled water to produce said solution of extraction including both HA and undesired proteins;
removing undesired proteins from said solution of extraction by mixing with sodium chloride and chloroform to provide a suspension;
precipitating said suspension using ethanol, and removing the liquid fraction;
at least partially dehydrating the solid fraction of said suspension;
re-hydrating said solid fraction using an aqueous solution of sodium chloride;
removing proteins by use of chloroform at a PH in the range of about 4.5 to about 5.0 to provide a suspension including HA;
performing enzymatic decomposition on said suspension to provide a decomposition suspension including HA having a desired molecular weight range;
precipitating said decomposition suspension to provide a compound of precipitations;
ionizing said compound of precipitations using an aqueous solution of sodium chloride to provide a solution of ionization;
precipitating said solution of ionization using ethanol to provide a precipitation; and
dehydrating said precipitation to provide dry sodium hyaluronate powder having said desired molecular weight range.

5. A dry sodium hyaluronate powder produced according to the method of claim 4.

6. The method of claim 4 further including the step of producing said HA with said desired average molecular weight range from about 8,000 Daltons to about 15,000 Daltons.

7. The method of claim 6 further including the step of producing said HA with said desired average molecular weight range from about 8,000 Daltons to about 12,000 Daltons.

8. The method of claim 7 further including the step of producing said HA with said desired average molecular weight range from about 8,000 Daltons to about 10,000 Daltons.

9. The method of claim 4 wherein said providing of said HA in a desired form further includes the step of packaging said sodium hyaluronate powder in gelatin capsules each containing from about 50 mg to about 250 mg of said sodium hyaluronate powder.

10. The method of claim 9 further including the step of packaging said sodium hyaluronate powder in gelatin capsules each containing from about 100 mg to about 200 mg of said sodium hyaluronate powder.

11. The method of claim 10 further including the step of packaging said sodium hyaluronate powder in gelatin capsules each containing about 150 mg of said sodium hyaluronate powder.

12. The method of claim 1 including the step of utilizing chicken hen's comb.

13. The method of claim 12 further including the steps of:
utilizing frozen, ground hen's comb;
hydrating said frozen, ground hen's comb using an aqueous solution including enzyme;
heating the aqueous solution including enzyme and hydrated hen's comb to a temperature and for a time sufficient to simultaneously perform both extraction of HA and undesired proteins from the hen's comb, while also effecting enzymatic decomposition of the extracted HA to said desired low molecular weight range, providing said solution of extraction;
removing undesired proteins from said solution of extraction to provide a solution substantially free of undesired proteins;
purifying said solution substantially free of undesired proteins to provide a purified solution; and
dehydrating said purified solution to provide a dried salt of HA having said desired average molecular weight range.

14. The method of claim 13 including the steps of milling said dried salt of HA, and sieving the milled dried salt of HA, to provide a dry powder form of HA salt having said desired average molecular weight range.

15. A dry powder form of HA produced according to the method of claim 14.

16. A method of producing Hyaluronic acid (HA) in the form of a dry powdered salt of HA with a desired low average molecular weight range from about 8,000 to about 15,000 Daltons which is completely free of bleach and is easily assimilated by humans via the digestive tract, and of providing said HA to a human for beneficial human oral consumption as a food supplement, said method comprising steps of:
providing a solution of extraction including HA and undesired proteins;
removing a substantial fraction of said undesired proteins from said solution of extraction;
decomposing the HA to a desired low molecular weight range without the use of bleach, thus allowing for beneficial human oral consumption and utilization of said HA;
separating said decomposed HA from solution; and
drying the decomposed HA to a dry powder form for oral human consumption; and
providing the dry powder HA in a desired packaging form to a human for oral consumption of said HA as a food supplement which is completely free of bleach.

17. The method of claim 16 further including the steps of
utilizing dehydrated, granulated rooster comb;
soaking said dehydrated, granulated rooster comb in substantially only distilled water to produce said solution of extraction including both HA and undesired proteins;
removing undesired proteins from said solution of extraction by mixing with sodium chloride and chloroform to provide a suspension;
precipitating said suspension using ethanol, and removing the liquid fraction;
at least partially dehydrating the solid fraction of said suspension;
re-hydrating said solid fraction using an aqueous solution of sodium chloride;
removing proteins from said re-hydrated solid fraction by use of chloroform at a PH in the range of about 4.5 to about 5.0 to provide a suspension including HA;
performing enzymatic decomposition on said suspension to provide a decomposition suspension including HA having said desired average molecular weight range;
precipitating said decomposition suspension to provide a compound of precipitations;
ionizing said compound of precipitations using an aqueous solution of sodium chloride to provide a solution of ionization;
precipitating said solution of ionization using ethanol to provide a precipitation; and
dehydrating said precipitation to provide dry sodium hyaluronate powder having said desired average molecular weight range.

18. A dry sodium hyaluronate powder produced according to the method of claim 17.

19. A method including providing to a human as a food supplement a dry sodium hyaluronate powder produced according to the method of claim 17.

20. A method of producing Hyaluronic acid (HA) in the form of a dry powdered salt of HA with a desired low average molecular weight range from about 8,000 to about 15,000 Daltons which is completely free of bleach and is easily assimilated by humans via the digestive tract, and of providing said HA to a human for beneficial human oral consumption as a food supplement, said method comprising steps of:
utilizing dehydrated, granulated rooster comb; and soaking said dehydrated, granulated rooster comb in substantially only distilled water to providing a solution of extraction including HA and undesired proteins;
removing a substantial fraction of said undesired proteins from said solution of extraction;
decomposing the HA to a desired low molecular weight range without the use of bleach, thus allowing for beneficial human oral consumption and utilization of said HA;
separating said decomposed HA from solution; and
drying the decomposed HA to a dry powder form for oral human consumption; and
providing the dry powder HA in a desired packaging form to a human for oral consumption of said HA as a food supplement which is completely free of bleach.
